(19) United States
(12) Patent Application Publication
(10) Pub. No.: US 2017/0027842 A1
(43) Pub. Date: Feb. 2, 2017

(54) ANTI-ULTRAVIOLET FLAKY CELLULOSE MATERIAL, PREPARATION METHOD AND USE THEREOF

(71) Applicant: Technical Institute of Physics and Chemistry of the Chinese Academy of Sciences, Beijing (CN)

(72) Inventors: Min Wu, Beijing (CN); Yong Huang, Beijing (CN); Mengmeng Zhao, Beijing (CN)

(21) Appl. No.: 15/303,101

(22) PCT Filed: Apr. 9, 2015
(86) PCT No.: PCT/CN2015/076157

§ 371 Date: Oct. 10, 2016

(30) Foreign Application Priority Data
Apr. 10, 2014 (CN) 201410143766.5

(51) Int. Cl. A61K 8/73; C09D 7/12; C08J 3/12; C09D 5/00; A61K 8/02; A61Q 17/04

(52) U.S. Cl. CPC A61K 8/731; A61K 8/0254; A61Q 17/04; C08J 3/12; C09D 5/00; C09D 7/1291; C09D 7/1283; A61K 2800/412; C08J 2301/02

(57) ABSTRACT

The present invention discloses an anti-ultraviolet flaky cellulose material which is characterized in that the cellulose material has a flaky morphology, a particle width of 5-200 μm and a thickness of 0.01-10 μm, and the flaky cellulose material can block transmission of ultraviolet ray within the range of UVA (320-400 nm) and UVB (280-320 nm). Meanwhile, the present invention also provides a preparation method of the flaky cellulose material and a use thereof.

### TECHNICAL FIELD

The present invention relates to the technical field of natural polymer materials, and more particularly relates to a flaky cellulose material with anti-ultraviolet function and a preparation method and a use thereof.

### BACKGROUND

In recent years, with the destruction of the ozone layer and the intensification of the sunspot activity, the ultraviolet dose radiated on the surface of the earth increases. Sunscreen products such as sunscreen cosmetics, sunscreen textiles and the like are more and more valued by people for avoiding damage caused by ultraviolet irradiation. The sunscreen effect of cosmetics is due to the absorption effect of the ultraviolet absorbent added in the cosmetics. However, with the role of sunscreen, the ultraviolet absorbent can screen also causes potential damage to human health such as skin injury, etc. For example, nano titanium dioxide known by people is currently the main additive of the sunscreen cosmetics. While, Titanium dioxide may also stimulate a free radical single-electron structure with ingredients of the sunscreen cosmetics under the irradiation of ultraviolet ray and visible light. This structure has a strong oxidizing ability and can cause damage to human skin cells.

Cellulose is the most abundant natural polymer in nature, which is mainly derived from plants. As an environment-friendly natural material, cellulose is non-toxic and harmless to human bodies, usually used as additive of food and drugs. The common morphology of cellulose materials is granular, fibrous, needlelike or rod-like. At present, there is no report on flaky cellulose nor its use as anti-ultraviolet material.

### BRIEF DESCRIPTION

The first technical problem to be solved in the present invention is to provide an anti-ultraviolet flaky cellulose material which can block transmission transmission of ultraviolet ray within a range of UVA (320-400 nm) and UVB (280-320 nm).

The second technical problem to be solved in the present invention is to provide a preparation method of the anti-ultraviolet flaky cellulose material.

The third technical problem to be solved in the present invention is to provide an application of the anti-ultraviolet flaky cellulose material.

To solve the above first technical problem, the present invention adopts the following technical solutions:

An anti-ultraviolet flaky cellulose material, which has a flaky morphology, with a particle width of 5-200 μm and a thickness of 0.01-10 μm. Because of special flaky structure, the cellulose material can block transmission of ultraviolet ray within the range of UVA (320-400 nm) and UVB (280-320 nm).

Preferably, the cellulose material is 30-50 μm in width and 0.01-10 μm in thickness. Preferably, the cellulose material is selected from natural cellulose or regenerated cellulose.

Preferably, the natural cellulose is selected from natural herbaceous plants or natural woody plants, and the regenerated cellulose is selected from one or more of viscose fiber, copper ammonia fiber, Tencel and alkali urea solution fiber.

To solve the above second technical problem, the present invention adopts the following technical solutions:

1) the cellulose material and a solid polymer abrasive material are mixed and milled; after milled, the morphology of the cellulose becomes flaky, thereby obtaining the flaky cellulose material; and

2) the flaky cellulose material obtained in step 1) is separated from the solid polymer abrasive material to obtain the anti-ultraviolet flaky cellulose material.

Preferably, the mixture in step 1) is the mixture of the solid polymer abrasive material and the cellulose material by weight ratio of 100-180:100, the milling is conducted in a ball mill or a grinding mill, the milling rate is 200-500 rpm, and the milling time is 2-30 h.

Preferably, the solid polymer abrasive material in step 1) is selected from polyolefin and a derivative and a copolymer thereof. Preferably, the solid polymer abrasive material is selected from one or more of polyethylene (PE), polypropylene (PP), polyvinyl chloride (PVC), polystyrene (PS), polyacrylonitrile, polyformaldehyde, epoxy resin, polyethylene terephthalate (PET), polymethyl methacrylate (PMMA), poly(butylene succinate) (PBS), polycarbonate (PC), poly(ε-caprolactone) (PCL), polylactic acid (PLA), polyamide, nylon 1010, polysulfone, bakelite, silicone rubber and fluoro alkylsilane (FAS).

Preferably, the cellulose material in step 1) is selected from natural cellulose or regenerated cellulose, the natural cellulose is selected from natural herbaceous plants or natural woody plants, and the regenerated cellulose is selected from one or more of viscose fiber, copper ammonia fiber, Tencel and alkali urea solution fiber.

Preferably, the separation of the flaky cellulose material from the solid polymer abrasive material in step 3) means separating the ground cellulose from the solid polymer abrasive material by using a sieve to make the width of the anti-ultraviolet flaky cellulose particle be 30-50 μm.

To solve the above third technical problem, the present invention adopts the following technical solutions:

The flaky cellulose material with an anti-ultraviolet function is applied to anti-ultraviolet cosmetics, anti-ultraviolet paint, anti-ultraviolet films, anti-ultraviolet fabric, etc.

The present invention has the following beneficial effects:

In the prevent invention, cellulose powder and the solid polymer abrasive material are mechanically milled to obtain a new flaky cellulose material, which is not discovered previously. The flaky cellulose material has a function of blocking transmission of ultraviolet ray.

### BRIEF DESCRIPTION OF THE DRAWINGS

The specific embodiments of the present invention will be further described below in detail in conjunction with the accompanying figures.

FIG. 1 is SEM of the flaky cellulose material of embodiment 1.

FIG. 2 is UV-VIS reflection spectra of the flaky cellulose of embodiment 1.

FIG. 3 is UV-VIS reflection spectrum of flaky cellulose of embodiment 2, embodiment 6 and titanium dioxide nanoparticles.

FIG. 4 is UV-VIS reflection spectra of the flaky cellulose of embodiment 5.

### DETAILED DESCRIPTION

To describe the present invention more clearly, the present invention is further described below in combination with the preferred embodiments and the figures. Those skilled in the art should understand that the contents which are specifically described below are illustrative, rather than restrictive, and shall not be regarded as limiting the protection scope of the present invention.

### Embodiment 1

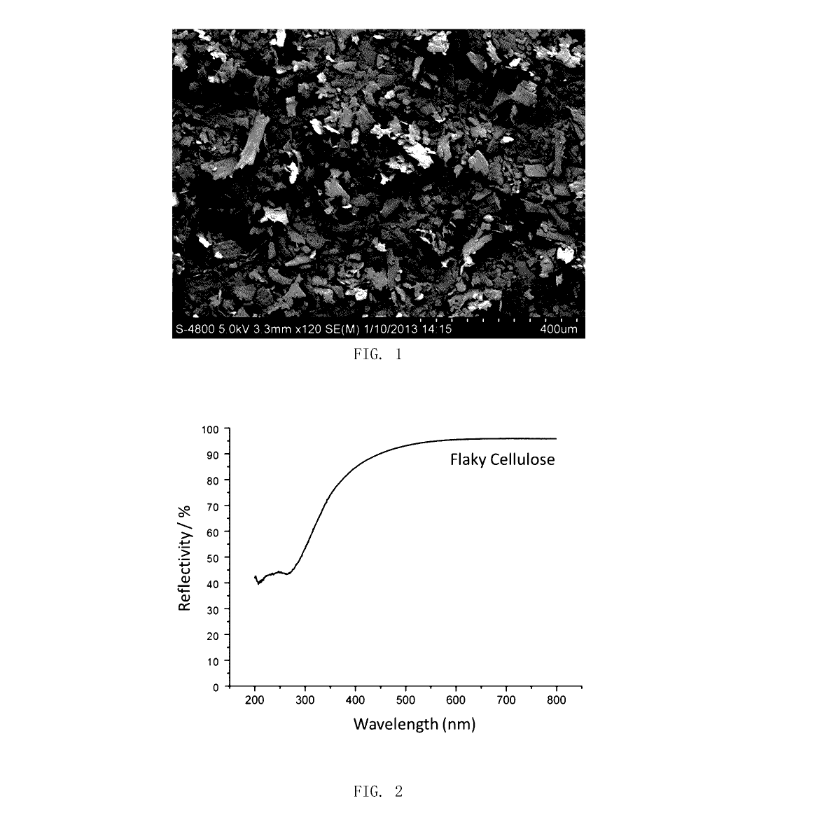

50 g of dry cellulose from a wood pulp is taken to be mixed with 50 g of polyethylene (PE) and mechanically milled for 16 hours. The milling balls are stainless steel balls with the diameter of 16 mm, and the grinding rate is 300 r/min. After milled, the cellulose powder and PE are separated by a stainless steel sieve to obtain the cellulose powder. The morphology of the obtained cellulose powder is flaky platelet, as shown in FIG. 1. The platelets are 40-80 μm wide 50-100 nm thick.

### Embodiment 2

50 g of dry cellulose from a wood pulp is taken to be mixed with 50 g of PE and mechanically milled for 4 h. The milling balls are stainless steel balls with the diameter of 16 mm, and the grinding rate is 500 r/min. After milled ground, the cellulose powder and PE are separated by a stainless steel sieve to obtain the cellulose powder. The morphology of the obtained cellulose powder is flaky platelet with the width of 80-200 μm and thickness of 1-5 μm.

### Embodiment 3

50 g of dry cellulose from a wood pulp is taken to be mixed with 50 g of polypropylene (PP) and mechanically milled for 28 h. The milling balls are stainless steel balls with the diameter of 16 mm, and the grinding rate is 500 r/min. After milled, the cellulose powder and PP are separated by a stainless steel sieve to obtain the cellulose powder. The morphology of the obtained cellulose powder is flaky platelet, with the width of 5-10 μm and thickness of 0.05-0.1 μm.

### Embodiment 4

30 g of dry cellulose from a cotton pulp is taken to be mixed with 50 g of PP and mechanically milled for 16 h. The grinding balls are stainless steel balls with the diameter of 16 mm, and the grinding rate is 300 r/min. After milled, the cellulose powder and PP are separated by a stainless steel sieve to obtain the cellulose powder. The morphology of the obtained cellulose powder is flaky platelet, with the width of 30-50 μm and thickness of 80-100 nm.

### Embodiment 5

20 g of dry copper ammonia regenerated cellulose is taken to be mixed with 30 g of polyvinyl chloride (PVC), and mechanically milled for 16 h. The grinding balls are a stainless steel balls with the diameter of 16 mm, and the grinding rate is 300 r/min. After milled, the cellulose powder and PVC are separated by a stainless steel sieve to obtain the cellulose powder. The morphology of the obtained cellulose powder is flaky platelet, with the width of 5-20 μm and thickness of 60-90 nm.

### Embodiment 6

30 g of dry microcrystalline cellulose is taken to be mixed with 50 g of PVC and mechanically milled for 16 h. The grinding balls are stainless steel balls with the diameter of 16 mm, and the grinding rate is 500 r/min. After milled, the cellulose powder and PVC are separated by a stainless steel sieve to obtain the cellulose powder. The morphology of the obtained cellulose powder is flaky platelet, with the width of 2-6 μm and thickness of 0.02-0.06 μm.

### Embodiment 7

The flaky cellulose obtained in embodiments 1-6 shows good anti-ultraviolet capability in UVA (320-400 nm) and UVB (280-320 nm). FIG. 2 is the UV-VIS reflection spectra of the flaky cellulose of embodiment 1. The flaky cellulose shows a reflectivity up to 85% against UVA and more than 50% against UVB, and almost total reflection against visible light. The 50 g of flaky cellulose is used for replacing common sunscreen additives such as nano titanium dioxide, octyl methoxycinnamate, benzophenone-3, etc. to be added to ingredients of the cosmetic ointment matrix formula: 100 g of white vaseline, 250 g of liquid paraffin, 1 g of mint, 50 g of dimethylcyclosiloxane, 50 g of dimethyl siloxane, 100 g of glycerin, 2 g of sodium bisulfite, 2 g of sorbic acid, 140 g of emulsifier and 1000 g of distilled water. The content of the cellulose is 5%. The sun protection factor (SPF) value of the prepared cosmetic is higher than 30. The sunscreen cosmetic has sunscreen effect due to the reflection of the flaky cellulose against ultraviolet ray, which belongs to a mechanism of physical sunscreen, and has good capability of covering skin blemishes due to total reflection within the range of visible light. In addition, as a cosmetic additive, the flaky cellulose gives fine and smooth feel during use due to flaky morphology, better than other granular cosmetics. The sunscreen paint can be prepared by adding the flaky cellulose into the paint by the weight ratio of 10%, which can also be made into a film as a sunscreen film. Besides, the flaky cellulose can be added into the fabric to make sunscreen clothes and textiles.

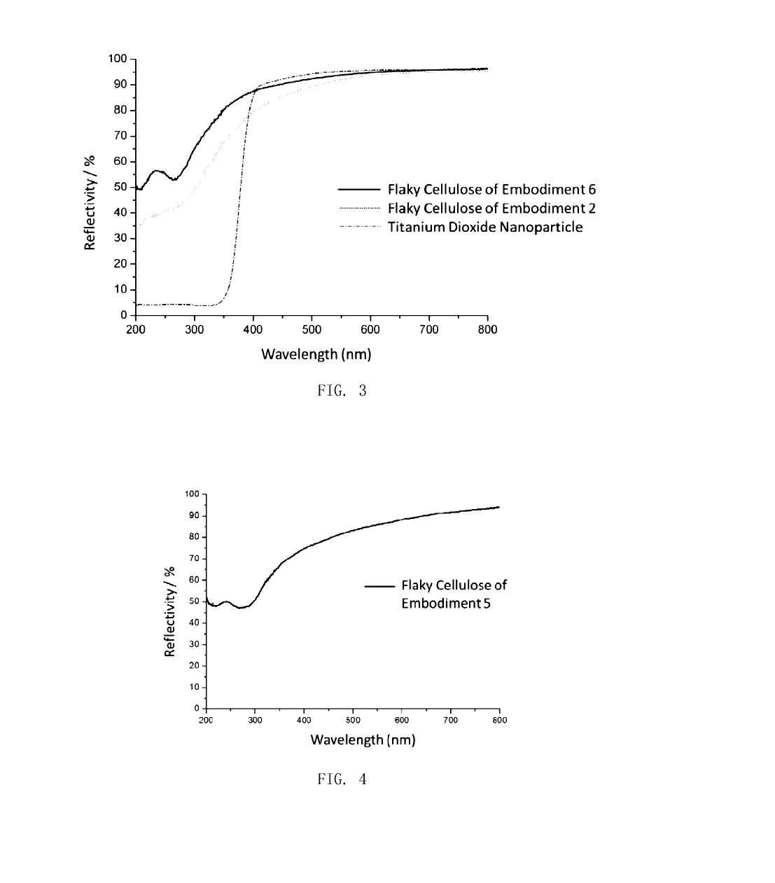

FIG. 3 is the reflection spectrum of flaky cellulose of embodiment 2, embodiment 6 and titanium dioxide nanoparticles. FIG. 4 is the reflection spectra of the flaky cellulose of embodiment 5. It can be seen from the above figures that although the size of the obtained flaky cellulose varies due to different raw materials of the cellulose, the reflection of the flaky cellulose against UVA, UVB and visible light is slightly different. The ultraviolet reflectivity of the flaky cellulose obtained in embodiments 1-6 is much higher than that of titanium dioxide nanoparticles, as shown in FIG. 3.

It is apparent that the above embodiments of the present invention are merely examples given for clearly illustrating the present invention, not for limiting the embodiments of the present invention. For those technical personnel in the field, different forms of other variations or changes can also be made based on the above description. The embodiments are not exhaustive herein. Apparent variations or changes derived from the technical solution of the present invention still belong to the protection scope of the present invention.

1. An anti-ultraviolet flaky cellulose material, wherein the cellulose material has a flaky morphology, a particle width of 5-200 μm and a thickness of 0.01-10 μm, and the cellulose material can block transmission of ultraviolet ray within a range of UVA (320-400 nm) and UVB (280-320 nm).

2. The anti-ultraviolet flaky cellulose material according to claim 1, wherein the particle width of the cellulose material is 30-50 μm.

3. The anti-ultraviolet flaky cellulose material according to claim 1, wherein the cellulose is selected from natural cellulose or regenerated cellulose.

4. The anti-ultraviolet flaky cellulose material according to claim 3, wherein the natural cellulose is selected from natural herbaceous plants or natural woody plants, and the regenerated cellulose is selected from one or more of viscose fiber, copper ammonia fiber, Tencel and alkali urea solution fiber.

5. A preparation method of the anti-ultraviolet flaky cellulose material according to claim 1, comprising the following steps:
1) mixing the cellulose material and the solid polymer abrasive material, wherein, after milled, the morphology of the cellulose becomes flaky, thereby obtaining the flaky cellulose material; and
2) separating the flaky cellulose material obtained in step 1) from the solid polymer abrasive material to obtain the anti-ultraviolet flaky cellulose material.

6. The method according to claim 5, wherein the mixing in step 1) is to mix the solid polymer abrasive material and the cellulose material by the weight ratio of 100-180:100, and wherein the milling is conducted in a ball mill or a grinding mill, the milling rate is 200-500 rpm, and the milling time is 2-30 hours.

7. The method according to claim 5, wherein the solid polymer abrasive material in step 1) is selected from polyolefin and a derivative and a copolymer thereof; and preferably, the solid polymer abrasive material is selected from one or more of polyethylene, polypropylene, polyvinyl chloride, polystyrene, polyacrylonitrile, polyformaldehyde, epoxy resin, polyethylene terephthalate, polymethyl methacrylate, poly(butylene succinate), polycarbonate, poly(ε-caprolactone), polylactic acid, polyamide, nylon 1010, polysulfone, bakelite, silicone rubber and fluoro alkylsilane.

8. The method according to claim 5, wherein the cellulose material in step 1) is selected from natural cellulose or regenerated cellulose, the natural cellulose is selected from natural herbaceous plants or natural woody plants, and the regenerated cellulose is selected from one or more of viscose fiber, copper ammonia fiber, Tencel and alkali urea solution fiber.

9. The method according to claim 5, wherein the separating the flaky cellulose material from the solid polymer abrasive material in step 3) is to separate the milled cellulose from the solid polymer abrasive material by using a sieve to make the particle width of the anti-ultraviolet flaky cellulose material be 30-50 μm.

10. A use of the anti-ultraviolet flaky cellulose material according to claim 1 in anti-ultraviolet cosmetics, anti-ultraviolet paint, anti-ultraviolet films and anti-ultraviolet fabric.

11. A use of the anti-ultraviolet flaky cellulose material according to claim 2 in anti-ultraviolet cosmetics, anti-ultraviolet paint, anti-ultraviolet films and anti-ultraviolet fabric.

12. A use of the anti-ultraviolet flaky cellulose material according to claim 3 in anti-ultraviolet cosmetics, anti-ultraviolet paint, anti-ultraviolet films and anti-ultraviolet fabric.

13. A use of the anti-ultraviolet flaky cellulose material according to claim 4 in anti-ultraviolet cosmetics, anti-ultraviolet paint, anti-ultraviolet films and anti-ultraviolet fabric.

14. A use of the anti-ultraviolet flaky cellulose material according to claim 5 in anti-ultraviolet cosmetics, anti-ultraviolet paint, anti-ultraviolet films and anti-ultraviolet fabric.

15. A use of the anti-ultraviolet flaky cellulose material according to claim 6 in anti-ultraviolet cosmetics, anti-ultraviolet paint, anti-ultraviolet films and anti-ultraviolet fabric.

16. A use of the anti-ultraviolet flaky cellulose material according to claim 7 in anti-ultraviolet cosmetics, anti-ultraviolet paint, anti-ultraviolet films and anti-ultraviolet fabric.

17. A use of the anti-ultraviolet flaky cellulose material according to claim 8 in anti-ultraviolet cosmetics, anti-ultraviolet paint, anti-ultraviolet films and anti-ultraviolet fabric.

18. A use of the anti-ultraviolet flaky cellulose material according to claim 9 in anti-ultraviolet cosmetics, anti-ultraviolet paint, anti-ultraviolet films and anti-ultraviolet fabric.
